(19) United States
(12) Patent Application Publication
(10) Pub. No.: US 2019/0359905 A1
(43) Pub. Date: Nov. 28, 2019

(54) DISTILLATE BLENDING SYSTEM WITH ONLINE DERIVED AND/OR INDICATED CETANE NUMBER ANALYZER AND RELATED METHODS

(71) Applicant: ExxonMobil Research and Engineering Company, Annandale, NJ (US)

(72) Inventors: Eric J. Lanni, Spring, TX (US); Cody R. Cole, Humble, TX (US); Arnoldus L. M. Van Adrichem, Vlaardingen (NL)

(21) Appl. No.: 16/412,770

(22) Filed: May 15, 2019

Related U.S. Application Data
(60) Provisional application No. 62/676,366, filed on May 25, 2018.

(51) Int. Cl. C10L 10/12; G01N 33/28

(52) U.S. Cl. CPC C10L 10/12; G01N 33/2829; C10L 2290/24; C10L 1/231; C10L 2200/0446; C10L 2290/58; C10L 2290/141; C10L 2270/026

(57) ABSTRACT

A method can include: mixing two or more feedstocks to produce a distillate product; optionally adding a cetane improver to the distillate product; collecting the distillate product in a tank; extracting a distillate product sample from the distillate product after mixing and before collecting in the tank; measuring a derived cetane number and/or a indicated cetane number (DCN/ICN) for the distillate product sample with an online DCN/ICN analyzer; communicating the DCN/ICN to a plant distributed control system; calculating an integrated DCN/ICN for the cumulative distillate product in the tank based on the measured DCN/ICN, previously measured DCN/ICN for portions of the distillate product in the tank, and process variables related to the mixing and cetane improver; and adjusting one or more of the process variables based on the integrated DCN/ICN.

### CROSS REFERENCE TO RELATED APPLICATIONS

This application claims priority to U. S. Provisional Application Ser. No. 62/676,366 filed May 25, 2018 which is herein incorporated by reference in its entirety.

### FIELD

This application relates to a system and method for improved control of the cetane number (CN) of distillate products during production via online measurement of a closely related and correlated property, derived cetane number (DCN).

### BACKGROUND

Cetane number (CN) is a key property of commercial diesel fuels that indicates the quality of fuel combustion by auto-ignition in a diesel engine. Higher CNs indicate superior combustion quality (optimum ignition delay following injection), and diesel fuels are manufactured to meet or exceed a minimum CN specification. Diesel fuels with “off-spec” (also known as “sub-specification”) CNs can either be reprocessed through expensive methods or downgraded to a lower product value. Additionally, excessive CNs result in value “give away” by the manufacturer as product quality exceeds requirement but receives the same commercial price. Thus, one main objective in diesel fuel production is to set and meet tight CN targets, which minimizes give away while also keeping a product batch safely “on-spec.”

Modern refineries produce diesel fuel through a blending process whereby liquid feedstocks, each with an independent CN and other qualities, are combined continuously online in a mixer, blend header, or straight run pipe. A single product stream is output to storage tank or pipeline for commercial distribution. One or more chemical additives may also be mixed into the product stream during blending in order to modify fuel qualities. Such additives include cetane improvers (e.g., alkyl nitrate compounds or peroxide compounds) used to boost the CN. Periodic measurement of various fuel qualities and blending conditions (e.g., feedstock, additive, and/or product stream flow rates, temperatures, and/or tank levels) provide input to a computer control system running a blend control application that can automatically adjust the blending process and parameters in order to optimize the quality of the final fuel product including the CN and other qualities (e.g., cloud point, sulfur content, and color).

Blend control generally improves with shorter measurement delay time (i.e., the delay between sampling of the process stream and output of an associated measurement) and faster input frequency because it more closely represents the instantaneous stream quality. However, off-line laboratory analyses typically involve multiple hours of delay time because the analytical methods have several manual steps (e.g., sample acquisition, transportation, manual analysis, and data entry).

The current primary test method for the CN (according to ASTM D975-18, “Standard Specification for Diesel Fuel Oils”) is offline test ASTM D613-18 that utilizes the “cetane engine,” an instrumented, variable-compression ratio diesel engine laboratory apparatus. The cetane engine is expensive to purchase and maintain, slow to generate measurements (˜1 hr), and requires significant manual intervention by an expert operator throughout the process. Due to the heavily manual, operator-dependent method, this CN measurement technique suffers from poor inter-lab reproducibility and has not been successfully automated for online measurement, although the concept has been disclosed in US Pat. No. 6,155,101.

Alternatively, CN can be approximated by a cetane index (CI) calculated from two online measurements (1) product density and (2) distillation points according to methods such as ASTM D4737-10(2016), ASTM D976-06(2016), and ISO 4264:2007. Importantly however, CI does not account for cetane improver effect, so CI cannot be used to directly predict and control final distillate product CN quality if improver is used.

More recently, a derived cetane number (DCN) has been introduced as an alternative measurement to CN and CI. Several similar lab analyzers and the associated DCN methods (e.g., ASTM D7668-17, ASTM D6890-16e1, and ASTM D7170-16) have been approved as alternatives to the cetane engine test for CN measurement according to ASTM D975-18. Compared with the cetane engine test, these analyzers utilize a constant volume combustion chamber (CVCC) to determine DCN from measurement of chamber pressure following fuel sample injection. The CVCC apparatus and method are simpler, more robust, more automated, and more precise than the cetane engine, demonstrating superior precision (repeatability and inter-lab reproducibility). However, all reported and commercialized DCN analyzers are strictly offline, laboratory devices that take time, sometimes hours, and do not approximate the current stream quality after the measurements are complete.

Considering the aforementioned CN, DCN, and CI measurement approaches currently available, there exists a need and opportunity for a fast, reliable, online method and system of DCN measurement and control for the distillate product blending process.

### SUMMARY

A method can comprise: mixing two or more feedstocks to produce a distillate product; optionally adding a cetane improver to the distillate product; collecting the distillate product in a tank; extracting a distillate product sample from the distillate product before collecting in the tank; measuring a derived cetane number and/or a indicated cetane number (DCN/ICN) for the distillate product sample with an online DCN/ICN analyzer; communicating the DCN/ICN to a plant distributed control system; calculating an integrated DCN/ICN for the cumulative distillate product in the tank based on the measured DCN/ICN, previously measured DCN/ICN for portions of the distillate product in the tank, and process variables related to the mixing and cetane improver; and adjusting one or more of the process variables based on the integrated DCN/ICN.

A method can comprise: mixing two or more feedstocks to produce a distillate product; optionally adding a cetane improver to the distillate product; transporting the distillate product to a pipeline; extracting a distillate product sample from the distillate product before transporting to the pipeline; measuring a derived cetane number and/or a indicated cetane number (DCN/ICN) for the distillate product sample with an online DCN/ICN analyzer; communicating the DCN/ICN to a plant distributed control system; calculating a current DCN/ICN for the distillate product being transported to the pipeline based on the measured DCN/ICN and process variables related to the mixing and cetane improver; and adjusting one or more of the process variables based on the current DCN/ICN and a target value and/or a threshold range for the DCN/ICN.

A distillate blending system can comprise: a supply of two or more feedstocks; a mixer that receives and mixes the two or more feedstocks to produce a distillate product; a processing line that transports the distillate product to a tank or a pipeline; an online distillate product analysis unit comprising an online derived cetane number analyzer and/or an online indicated cetane number analyzer (an online DCN/ICN analyzer) fluidly connected to the processing line for extraction of a distillate product sample from one or more locations along the processing line; a cetane improver injection along the processing line; and a plant distributed control system in electronic communication with at least the online DCN/ICN analyzer.

### BRIEF DESCRIPTION OF THE DRAWINGS

The following figures are included to illustrate certain aspects of the embodiments, and should not be viewed as exclusive embodiments. The subject matter disclosed is capable of considerable modifications, alterations, combinations, and equivalents in form and function, as will occur to those skilled in the art and having the benefit of this disclosure.

FIG. 1 illustrates a diagram of a distillate blending system with an online DCN analyzer.

FIG. 2 is a diagram of a first example method of operation for a distillate blending system with an online DCN analyzer.

FIG. 3 is a diagram of a second example method of operation for a distillate blending system with an online DCN analyzer.

FIG. 4 is a diagram of a third example method of operation for a distillate blending system with an online DCN analyzer.

### DETAILED DESCRIPTION

This application relates to a system and method for improved control of the cetane number (CN) of distillate products during production via online measurement of a closely related and correlated property(s), derived cetane number (DCN) and/or indicated cetane number (ICN). DCN and ICN are similarly measured values where DCN is defined by correlation equation established using calibration reference material(s) of known CN and ignition and/or combustion delays, while ICN is determined using calibration curve defined by blended primary reference samples. DCN measurement details can be found in ASTM D6890-16e1, ASTM D7170-16, ASTM D7668-17, and U.S. Pat. No. 7,949,471. ICN measurement details can be found in DIN EN 17155 (2017).

### Distillate Blending System

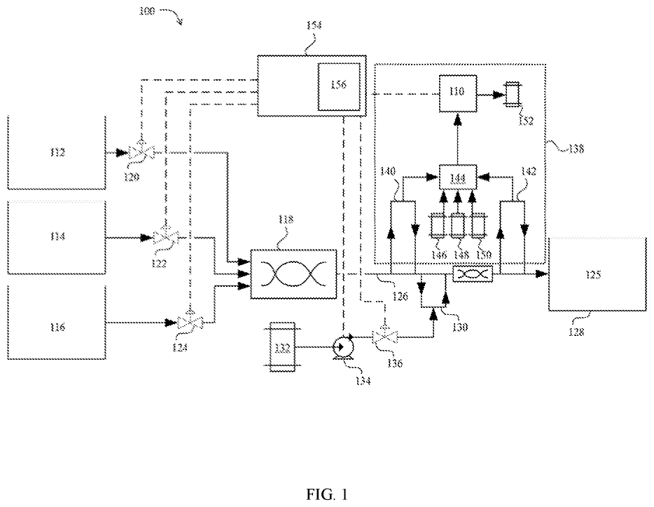

FIG. 1 illustrates a diagram of a distillate blending system 100 with an online DCN/ICN analyzer 110. As used herein, a term modified by “DCN/ICN” refers to DCN and/or ICN. For example, online DCN/ICN analyzer 110 can be an online analyzer that measures and/or determines only DCN, only ICN, or both DCN or ICN.

In the illustrated distillate blending system 100, three feedstocks 112, 114, 116 are metered into a mixer/blender 118 to produce a distillate product 125. The metering of the feedstocks 112, 114, 116 is controlled by corresponding control valves 120, 122, 124. The distillate product 125 is passed through a processing line 126 where cetane improver can optionally be mixed into the distillate product 125 before being collected in a tank 128 for storage. Alternatively, the tank 128 could be replaced with a pipeline for distributing the distillate product 125.

Examples of feedstocks include, but are not limited to, biodiesel, conventional diesel, butane, reformate, light fuel oil or light cycle oil (e.g., from fluid catalytic cracking), heavy fuel oil (e.g., from fluid catalytic cracking), alkylate fuel, virgin distillates (i.e., crude unit side streams including atmospheric pipestill and vacuum pipestill rundowns), kerosene, jet fuels, light coker gas oil (LKGO), hydrocracker unit outputs (e.g., side streams and bottoms), fatty acid methyl esters (FAME), hydrogenated vegetable oils (HVO), gas-to-liquids (GTL) materials, and combinations thereof.

Examples of cetane improvers include, but are not limited to, alkyl nitrate-type cetane improvers (e.g., ethylhexylnitrate) and peroxide-type cetane improvers (e.g., di-tert-butyl-peroxide (DTBP)).

Along the processing line 126 is a cetane improver injection flow 130 where the cetane improver is supplied from a cetane improver supply 132 via a cetane improver injection pump 134. The cetane improver injection pump 134 and a control valve 136 control how much cetane improver is supplied via the cetane improver injection flow 130. After the cetane improver is injected into the distillate product 125, the mixture is passed through a mixer to ensure homogeneous distribution of the cetane improver in the distillate product 125.

Along the processing line 126 is also an online distillate product analysis unit 138 that, as illustrated, includes at least one distillate product extraction circuit 140 before cetane improver injection, a distillate product extraction circuits 142 after cetane improver injection, a sample handling system 144, the online DCN/ICN analyzer 110, a calibration standard 146, a validation standard 148, a cleaning fluid 150, and a waste vessel 152. One or more samples of the distillate product 125 can be extracted from the processing line 126 before and/or after cetane improver injection using the extraction circuits 140, 142. Each of the distillate product samples is then physically transported and conditioned by the sample handling system 144 to be suitable for analysis by the online DCN/ICN analyzer 110. When samples are extracted before and after cetane improver injection, they are analyzed separately, where the analysis of each can be compared. Any portion of the distillate product sample not used for DCN/ICN analysis can be returned to the processing line 126. Alternatively, such distillate product sample could be collected in waste containers.

The online distillate product analysis unit 138 also includes one or more calibration standards 146 for calibrating the online DCN/ICN analyzer 110, one or more validation standards 148 for testing the calibration of the online DCN/ICN analyzer 110, and one or more cleaning fluids 150 for cleaning the online DCN/ICN analyzer 110. The sample handling system 144 includes the necessary stream switching manifold, connections, valves, and pumps to switch between the various fluids to deliver the desired fluid to the online DCN/ICN analyzer 110. Fluids having been through the online DCN/ICN analyzer 110 are disposed of into the waste vessel 152 or back to the processing line 126.

The distillate blending system 100 also includes a plant distributed control system 154 having a non-transitory, computer-readable medium hosting a software 156. The plant distributed control system 154 communicates (wired and/or wireless) with various components of the distillate blending system 100 illustrated with dashed lines in FIG. 1.

While FIG. 1 illustrates communication between the plant distributed control system 154 and the control valves 120, 122, 124, 136, the online DCN/ICN analyzer 110, and the cetane improver injection pump 134, some communications may be removed and/or additional communications can be added. For example, a component of the distillate blending system 100 may require manual adjustment by an operator rather than computer-controlled adjustment by the plant distributed control system 154. Such operator(s) may be on-site, off-site, or a combination thereof

By having the online DCN/ICN analyzer 110, the distillate blending system 100 can be operated by a variety of methods to control the quality of the distillate product 125.

### Example Method 1—Input to Blend Control

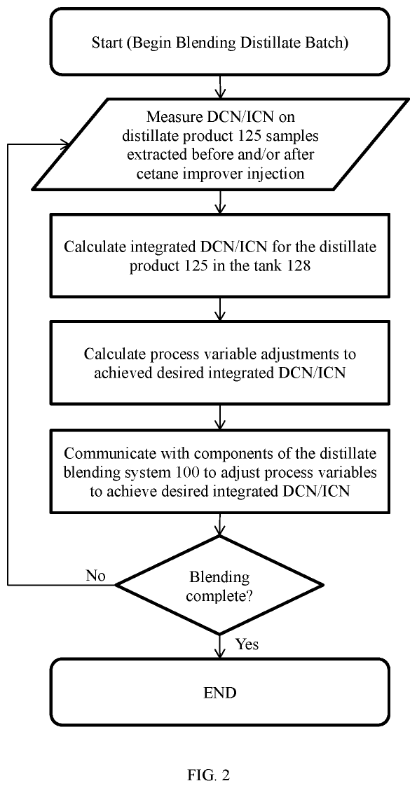

FIG. 2 is a diagram of a first example method of operation for a distillate blending system with an online DCN/ICN analyzer. With continued reference to FIG. 1, in the first example method of operation, the online DCN/ICN analyzer 110 automatically and periodically transmits DCN/ICN measurements of samples extracted from before and/or after the cetane improver injection to the plant distributed control system 154. The software 156 estimates an integrated DCN/ICN for the distillate product 125 in the tank 128 based on the DCN/ICN measurements, other process variables, and historic DCN/ICN measurements for the distillate product 125 already in the tank 128. Then, the software 156 adjusts the process variables to produce more distillate product 125 that when mixed with the distillate product 125 in the tank 128 would achieve the target DCN/ICN for the entire distillate product batch.

This method minimizes the discrepancy between the DCN/ICN of the final distillate product 125 and desired DCN/ICN for the entire distillate product batch.

Process variables can include, but are not limited to, other chemical properties, physical properties, flow rates, and/or tank levels relating to feedstocks 112, 114, 116, distillate product 125 at various points along the processing line 126, and additives including cetane improver. Specific examples of process variables include, but are not limited to, a composition of each of the two or more feedstocks, a relative concentration ratio of the two or more feedstocks, a flow rate of each of the two or more feedstocks, an available supply of each of the two or more feedstocks, a cetane improver injection rate, a cetane improver composition, a cetane improver concentration in the distillate product, an available supply of the cetane improver, a current composition and/or property of the distillate product 125 (e.g., CN, DCN, ICN, and/or other property), a desired composition and/or property of the distillate product 125 (e.g., CN, DCN, ICN, and/or other property), a heel volume in the tank 128, a heel composition and/or property in the tank 128 (e.g., CN, DCN, ICN, and/or other property), a current tank 128 fill level (in volume or percentage of capacity), a current tank 128 composition and/or property (e.g., CN, DCN, ICN, and/or other property), and combinations thereof. The “heel” in a tank is the fluid in the tank remaining after the previous discharge. For example, after filling tank 128 to a desired volume with a first distillate product, the tank 128 may be at least partially emptied. The first distillate product remaining in the tank 128 is the heel. The composition, properties, and/or volume of the heel can then be considered in the methods herein when producing a second distillate product.

Inputs may be measured directly from process or in a laboratory, inferred from other such measurements, or manually entered. In this example, the software 156 adjusts process variables in response to the DCN/ICN measurements by communicating with components of the distillate blending system 100 (e.g., the control valves 120, 122, 124, 136, the online DCN/ICN analyzer 110, and the cetane improver injection pump 134). The software 156 itself can be variable depending on the hardware of the online DCN/ICN analyzer 110. However, generally, the software 156 incorporates one or more formulas for determining DNC, ICN, or both or derivatives of those formulas. Generally, input for all calculations is one or more elapsed time measurements to particular pressure setpoints (e.g., injection delay, combustion delay) following sample injection into the constant volume combustion chamber. These elapsed time measurements are then used to determine DCN/ICN by empirical formula. Examples of such formulas can be found in ASTM D6890-16e1, ASTM D7170-16, ASTM D7668-17, U.S. Pat. No. 7,949,471, and DIN EN 17155 (2017), each of which are incorporated herein by reference. It should be noted that the equation used in the software 156 can be from previous or updated version of the foregoing ASTM and DIN EN standards and from any newly developed standards or formulas. EQ. 1 is a specific example from ASTM D6890-16e1.

DCN=4.460+186.6/ID EQ. 1

where DCN=derived cetane number result and ID=ignition delay (ms), which defined as “that period of time, in milliseconds (ms), between the start of fuel injection and the start of combustion as determined using the specific combustion analyzer applicable for the test method.”

In this example, all or most of the process variables are monitored and controlled by the plant distributed control system 154. Alternatively, only one to three process variables may be monitored and controlled by the plant distribution control system 154.

### Example Method 2—Closed-Loop Additive Control

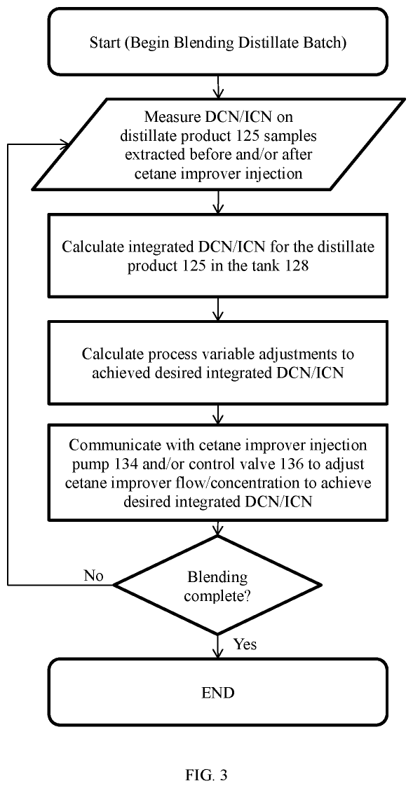

FIG. 3 is a diagram of a second example method of operation for a distillate blending system with an online DCN/ICN analyzer. The second example method is similar to the first example method but only one process variable is controlled by the plant distribution control system 154, the cetane improver flow/concentration. With continued reference to FIG. 1, the second example method of operation includes the online DCN/ICN analyzer 110 automatically and periodically transmitting DCN/ICN measurements of samples extracted from before and/or after the cetane improver injection to the plant distributed control system 154. The software 156 estimates an integrated DCN/ICN for the distillate product 125 in the tank 128 based on the DCN/ICN measurements, the cetane improver flow/concentration, and historic DCN/ICN measurements for the distillate product 125 already in the tank 128. Then, the software 156 communicates with the cetane improver injection pump 134 and/or the controller 136 to adjust the cetane improver flow/concentration to produce more distillate product 125 so that when mixed with the distillate product 125 in the tank 128 the target DCN/ICN for the entire distillate product batch is achieved.

Like the first example method, this method minimizes the discrepancy between the DCN/ICN of the final distillate product 125 and desired DCN/ICN for the entire distillate product batch. However, in contrast to the first example method, this method reduces complexity of the calculations by the software 156.

In both the first and second example methods, the software 156 may also include calculations that quantify process variable responses in terms of CN, DCN, and/or ICN change to final product. Such calculations can linearize the relation between cetane improver amount and CN, DCN, and/or ICN improvement in distillate product batch, which may include terms to account for the specific cetane improver compound(s) being used as well as each feedstock's 112, 114, 116 effects on CN response.

The first and second example methods rely on analysis and control by the plant distributed control system 154. Alternatively, the analysis and/or control could be done by one or more operators.

### Example Method 3—Manual Blend/Improver Control

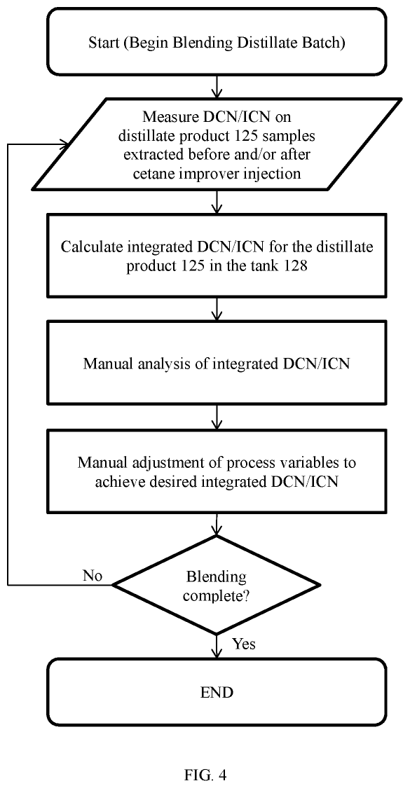

FIG. 4 is a diagram of a third example method of operation for a distillate blending system with an online DCN/ICN analyzer. This example is similar to the first example except that analysis and control is done manually. Manual control includes physically adjusting settings of components of the distillate blending system 100 (e.g., the control valves 120, 122, 124, 136) and inputting adjustments into plant distributed control system 154 to then be transmitted to the corresponding components of the distillate blending system 100.

With continued reference to FIG. 1, in the second example method of operation, the online DCN/ICN analyzer 110 automatically and periodically transmits DCN/ICN measurements of samples extracted from before and/or after the cetane improver injection to the plant distributed control system 154. The software 156 estimates an integrated DCN/ICN for the distillate product 125 in the tank 128 based on the DCN/ICN measurements, other process variables, and historic DCN/ICN measurements for the distillate product 125 already in the tank 128. Then, one or more operators analyze the integrated DCN/ICN and adjust the process variables (e.g., cetane improver flow/concentration and/or feedstock flow/ratios) to produce more distillate product 125 that when mixed with the distillate product 125 in the tank 128 would achieve the target DCN/ICN for the entire distillate product batch.

### ADDITIONAL EXAMPLES

Hybrids of the foregoing three examples are also encompassed in this disclosure. For example, the third example method could be modified such that the software 156 also performs an analysis of the integrated DCN/ICN and provides recommendations for process variable adjustments. Then, one or more operators can analyze the recommendations and manually adjust the process variables. In some instances, the software 156 may provide two or more recommendations that the operators may choose from. For example, changing either the relative concentration ratios of the feedstocks or changing the cetane improver flow/concentration could change the integrated DCN/ICN in a desired way. The software 156 may provide these two recommendations and the operators may choose which to proceed with based on other factors including, but not limited to, the supply of each feedstock, the supply of the cetane improver, the magnitude of the changes to the process variables, and combinations thereof

In other examples, any of the foregoing examples can be modified to include that the DCN/ICN measurements are triggered or requested by one or more operators in addition to or in place of the described automatic and periodic DCN/ICN measurements.

In yet other examples, any of the foregoing examples can be modified so that the distillate product 125 is not transferred to a tank but rather is conveyed to a pipeline. In such examples, the integrated DCN/ICN would be replaced with a current DCN/ICN measurement. The current DCN/ICN measurement may have a target value and/or a threshold range that should be met. Then, the software 156 and/or one or more operators may perform the described analyses and/or process variable adjustments to maintain the current DCN/ICN within the target value and/or the threshold range.

### Plant Distributed Control System

The plant distributed control system includes a computer system that comprises: a processor; and a tangible, machine-readable storage medium that stores machine-readable instructions for execution by the processor, the machine-readable instructions corresponding to one or more of the methods described herein. That is, the methods described herein can be performed on computing devices (or processor-based devices) that are part of the plant distributed control system and include a processor; a memory coupled to the processor; and instructions provided to the memory, wherein the instructions are executable by the processor to perform the methods described herein. The instructions can be a portion of code on a non-transitory computer readable medium. Any suitable processor-based device may be utilized for implementing all or a portion of embodiments of the present techniques, including without limitation personal computers, networks personal computers, laptop computers, computer workstations, mobile devices, multi-processor servers or workstations with (or without) shared memory, high performance computers, and the like. Moreover, embodiments may be implemented on application specific integrated circuits (ASICs) or very large scale integrated (VLSI) circuits.

The terms “non-transitory, computer-readable medium,” “tangible machine-readable medium,” or the like refer to any tangible storage that participates in providing instructions to a processor for execution. Such a medium may take many forms, including but not limited to, non-volatile media, and volatile media. Non-volatile media includes, for example, NVRAM, or magnetic or optical disks. Volatile media includes dynamic memory, such as main memory. Computer-readable media may include, for example, a floppy disk, a flexible disk, hard disk, magnetic tape, or any other magnetic medium, magneto-optical medium, a CD-ROM, any other optical medium, a RAM, a PROM, and EPROM, a FLASH-EPROM, a solid state medium like a holographic memory, a memory card, or any other memory chip or cartridge, or any other physical medium from which a computer can read. When the computer-readable media is configured as a database, it is to be understood that the database may be any type of database, such as relational, hierarchical, object-oriented, and/or the like. Accordingly, exemplary embodiments of the present techniques may be considered to include a tangible storage medium or tangible distributed medium and prior art-recognized equivalents and successor media, in which the software implementations embodying the present techniques are stored.

A transmission medium (e.g., for communications between the plant distributed control system 154 and other components of the distillate blending system 100 of FIG. 1) may be twisted wire pairs, coaxial cable, optical fiber, or some other suitable transmission medium, for transmitting signals such as electrical, optical, acoustical or other form of propagated signals (e.g., carrier waves, infrared signals, digital signals, etc.).

### EXAMPLES

Example 1: A method comprising: mixing two or more feedstocks to produce a distillate product; optionally adding a cetane improver to the distillate product; collecting the distillate product in a tank; extracting a distillate product sample from the distillate product after mixing and before collecting in the tank; measuring a derived cetane number and/or a indicated cetane number (DCN/ICN) for the distillate product sample with an online DCN/ICN analyzer; communicating the DCN/ICN to a plant distributed control system; calculating an integrated DCN/ICN for the cumulative distillate product in the tank based on the measured DCN/ICN, previously measured DCN/ICN for portions of the distillate product in the tank, and process variables related to the mixing and cetane improver; and adjusting one or more of the process variables based on the integrated DCN/ICN.

Example 2. The method of Example 1, wherein the process variables comprise one or more selected from the group consisting of: a composition of each of the two or more feedstocks, a relative concentration ratio of the two or more feedstocks, a flow rate of each of the two or more feedstocks, an available supply of each of the two or more feedstocks, a cetane improver injection rate, a cetane improver composition, a cetane improver concentration in the distillate product, an available supply of the cetane improver, a current composition and/or property of the distillate product, a desired composition and/or property of the distillate product, a heel volume in the tank, a heel composition and/or property in the tank, a current tank fill level, and a current tank composition and/or property.

Example 3. The method of Example 1 or 2, wherein the cetane improver is added to the distillate product after mixing and before collecting in the tank, and wherein the method further comprises: extracting a distillate product sample from the distillate product is after mixing and before adding the cetane improver to the distillate product.

Example 4. The method of Example 1 or 2, wherein the cetane improver is added to the distillate product after mixing and before collecting in the tank, and wherein the method further comprises: extracting a distillate product sample from the distillate product is after adding the cetane improver to the distillate product and before collecting in the tank.

Example 5. The method of Example 4, wherein the distillate product sample is a first distillate product sample and the DCN/ICN is a first DCN/ICN, and wherein the method further comprises: extracting a second distillate product sample from the distillate product is after mixing and before adding the cetane improver to the distillate product; measuring a second DCN/ICN for the second distillate product sample with the online DCN/ICN analyzer; and wherein adjusting one or more of the process variables is further based on the first and second DCN/ICNs.

Example 6. The method of any preceding Example, wherein calculating the integrated DCN/ICN and adjusting the one or more of the process variables is performed by a set of instructions on a non-transitory, computer-readable medium that is part of the plant distributed control system.

Example 7. The method of any preceding Example, wherein calculating the integrated DCN/ICN is performed by a set of instructions on a non-transitory, computer-readable medium that is part of the plant distributed control system; and adjusting the one or more of the process variables is performed by one or more operators.

Example 8. The method of any preceding Example, wherein adjusting the one or more of the process variables includes increasing a concentration of the cetane improver in the distillate product.

Example 9. The method of any preceding Example, wherein adjusting the one or more of the process variables includes changing a relative concentration ratio of the two or more feedstocks.

Example 10. A method comprising: mixing two or more feedstocks to produce a distillate product; optionally adding a cetane improver to the distillate product; transporting the distillate product to a pipeline; extracting a distillate product sample from the distillate product after mixing and before transporting to the pipeline; measuring a derived cetane number and/or a indicated cetane number (DCN/ICN) for the distillate product sample with an online DCN/ICN analyzer; communicating the DCN/ICN to a plant distributed control system; calculating a current DCN/ICN for the distillate product being transported to the pipeline based on the measured DCN/ICN and process variables related to the mixing and cetane improver; and adjusting one or more of the process variables based on the current DCN/ICN and a target value and/or a threshold range for the DCN/ICN.

Example 11. The method of Example 10, wherein the process variables comprise one or more selected from the group consisting of: a composition of each of the two or more feedstocks, a relative concentration ratio of the two or more feedstocks, a flow rate of each of the two or more feedstocks, an available supply of each of the two or more feedstocks, a cetane improver injection rate, a cetane improver composition, a cetane improver concentration in the distillate product, an available supply of the cetane improver, a current composition and/or property of the distillate product, and a desired composition and/or property of the distillate product.

Example 12. The method of Example 10 or 11, wherein the cetane improver is added to the distillate product after mixing and before transporting to the pipeline, and wherein the method further comprises: extracting a distillate product sample from the distillate product is after mixing and before adding the cetane improver to the distillate product.

Example 13. The method of Example 10 or 11, wherein the cetane improver is added to the distillate product after mixing and before transporting to the pipeline, and wherein the method further comprises: extracting a distillate product sample from the distillate product is after adding the cetane improver to the distillate product and before transporting to the pipeline.

Example 14. The method of Example 13, wherein the distillate product sample is a first distillate product sample and the DCN/ICN is a first DCN/ICN, and wherein the method further comprises: extracting a second distillate product sample from the distillate product is after mixing and before adding the cetane improver to the distillate product; measuring a second DCN/ICN for the second distillate product sample with the online DCN/ICN analyzer; and wherein adjusting one or more of the process variables is further based on the first and second DCN/ICNs.

Example 15. The method of any one of Examples 10-14, wherein calculating the current DCN/ICN and adjusting the one or more of the process variables is performed by a set of instructions on a non-transitory, computer-readable medium that is part of the plant distributed control system.

Example 16. The method of any one of Examples 10-15, wherein calculating the current DCN/ICN is performed by a set of instructions on a non-transitory, computer-readable medium that is part of the plant distributed control system; and adjusting one or more of the process variables is performed by one or more operators.

Example 17. The method of any one of Examples 10-16, wherein adjusting the one or more of the process variables includes increasing a concentration of the cetane improver in the distillate product.

Example 18. The method of any one of Examples 10-17, wherein adjusting the one or more of the process variables includes changing a relative concentration ratio of the two or more feedstocks.

Example 19. A distillate blending system comprising: a supply of two or more feedstocks; a mixer that receives and mixes the two or more feedstocks to produce a distillate product; a processing line that transports the distillate product to a tank or a pipeline; an online distillate product analysis unit comprising an online derived cetane number analyzer and/or an online indicated cetane number analyzer (an online DCN/ICN analyzer) fluidly connected to the processing line for extraction of a distillate product sample from one or more locations along the processing line; a cetane improver injection along the processing line; and a plant distributed control system in electronic communication with at least the online DCN/ICN analyzer.

Example 20. The distillate blending system of Example 19 wherein the one or more locations along the processing line for extraction of the distillate product sample includes a location upstream of the cetane improver injection and/or a location downstream of the cetane improver injection.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present invention. At the very least, and not as an attempt to limit the application of the doctrine of equivalents to the scope of the claim, each numerical parameter should at least be construed in light of the number of reported significant digits and by applying ordinary rounding techniques.

One or more illustrative embodiments incorporating the invention embodiments disclosed herein are presented herein. Not all features of a physical implementation are described or shown in this application for the sake of clarity. It is understood that in the development of a physical embodiment incorporating the embodiments of the present invention, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for those of ordinary skill in the art and having benefit of this disclosure.

While compositions and methods are described herein in terms of “comprising” various components or steps, the compositions and methods can also “consist essentially of” or “consist of” the various components and steps.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

1. A method comprising:
mixing two or more feedstocks to produce a distillate product;
optionally adding a cetane improver to the distillate product;
collecting the distillate product in a tank;
extracting a distillate product sample from the distillate product before collecting in the tank;
measuring a derived cetane number and/or a indicated cetane number (DCN/ICN) for the distillate product sample with an online DCN/ICN analyzer;
communicating the DCN/ICN to a plant distributed control system;
calculating an integrated DCN/ICN for the cumulative distillate product in the tank based on the measured DCN/ICN, previously measured DCN/ICN for portions of the distillate product in the tank, and process variables related to the mixing and cetane improver; and
adjusting one or more of the process variables based on the integrated DCN/ICN.

2. The method of claim 1, wherein the process variables comprise one or more selected from the group consisting of a composition of each of the two or more feedstocks, a relative concentration ratio of the two or more feedstocks, a flow rate of each of the two or more feedstocks, an available supply of each of the two or more feedstocks, a cetane improver injection rate, a cetane improver composition, a cetane improver concentration in the distillate product, an available supply of the cetane improver, a current composition and/or property of the distillate product, a desired composition and/or property of the distillate product, a heel volume in the tank, a heel composition and/or property in the tank, a current tank fill level, and a current tank composition and/or property.

3. The method of claim 1, wherein the cetane improver is added to the distillate product after mixing and before collecting in the tank, and wherein the method further comprises:
extracting a distillate product sample from the distillate product is after mixing and before adding the cetane improver to the distillate product.

4. The method of claim 1, wherein the cetane improver is added to the distillate product after mixing and before collecting in the tank, and wherein the method further comprises:
extracting a distillate product sample from the distillate product is after adding the cetane improver to the distillate product and before collecting in the tank.

5. The method of claim 4, wherein the distillate product sample is a first distillate product sample and the DCN/ICN is a first DCN/ICN, and wherein the method further comprises:
extracting a second distillate product sample from the distillate product is after mixing and before adding the cetane improver to the distillate product;
measuring a second DCN/ICN for the second distillate product sample with the online DCN/ICN analyzer; and
wherein adjusting one or more of the process variables is further based on the first and second DCN/ICNs.

6. The method of claim 1, wherein calculating the integrated DCN/ICN and adjusting the one or more of the process variables is performed by a set of instructions on anon-transitory, computer-readable medium that is part of the plant distributed control system.

7. The method of claim 1, wherein calculating the integrated DCN/ICN is performed by a set of instructions on a non-transitory, computer-readable medium that is part of the plant distributed control system; and adjusting the one or more of the process variables is performed by one or more operators.

8. The method of claim 1, wherein adjusting the one or more of the process variables includes increasing a concentration of the cetane improver in the distillate product.

9. The method of claim 1, wherein adjusting the one or more of the process variables includes changing a relative concentration ratio of the two or more feedstocks.

10. A method comprising:
mixing two or more feedstocks to produce a distillate product;
optionally adding a cetane improver to the distillate product;
transporting the distillate product to a pipeline;
extracting a distillate product sample from the distillate product before transporting to the pipeline;
measuring a derived cetane number and/or a indicated cetane number (DCN/ICN) for the distillate product sample with an online DCN/ICN analyzer;
communicating the DCN/ICN to a plant distributed control system;
calculating a current DCN/ICN for the distillate product being transported to the pipeline based on the measured DCN/ICN and process variables related to the mixing and cetane improver; and
adjusting one or more of the process variables based on the current DCN/ICN and a target value and/or a threshold range for the DCN/ICN.

11. The method of claim 10, wherein the process variables comprise one or more selected from the group consisting of: a composition of each of the two or more feedstocks, a relative concentration ratio of the two or more feedstocks, a flow rate of each of the two or more feedstocks, an available supply of each of the two or more feedstocks, a cetane improver injection rate, a cetane improver composition, a cetane improver concentration in the distillate product, an available supply of the cetane improver, a current composition and/or property of the distillate product, and a desired composition and/or property of the distillate product.

12. The method of claim 10, wherein the cetane improver is added to the distillate product after mixing and before transporting to the pipeline, and wherein the method further comprises:
extracting a distillate product sample from the distillate product is after mixing and before adding the cetane improver to the distillate product.

13. The method of claim 10, wherein the cetane improver is added to the distillate product after mixing and before transporting to the pipeline, and wherein the method further comprises:
extracting a distillate product sample from the distillate product is after adding the cetane improver to the distillate product and before transporting to the pipeline.

14. The method of claim 13, wherein the distillate product sample is a first distillate product sample and the DCN/ICN is a first DCN/ICN, and wherein the method further comprises:
extracting a second distillate product sample from the distillate product is after mixing and before adding the cetane improver to the distillate product;
measuring a second DCN/ICN for the second distillate product sample with the online DCN/ICN analyzer; and
wherein adjusting one or more of the process variables is further based on the first and second DCN/ICNs.

15. The method of claim 10, wherein calculating the current DCN/ICN and adjusting the one or more of the process variables is performed by a set of instructions on anon-transitory, computer-readable medium that is part of the plant distributed control system.

16. The method of claim 10, wherein calculating the current DCN/ICN is performed by a set of instructions on a non-transitory, computer-readable medium that is part of the plant distributed control system; and adjusting the one or more of the process variables is performed by one or more operators.

17. The method of claim 10, wherein adjusting the one or more of the process variables includes increasing a concentration of the cetane improver in the distillate product.

18. The method of claim 10, wherein adjusting the one or more of the process variables includes changing a relative concentration ratio of the two or more feedstocks.

19. A distillate blending system comprising:
a supply of two or more feedstocks;
a mixer that receives and mixes the two or more feedstocks to produce a distillate product;
a processing line that transports the distillate product to a tank or a pipeline;
an online distillate product analysis unit comprising an online derived cetane number analyzer and/or an online indicated cetane number analyzer (an online DCN/ICN analyzer) fluidly connected to the processing line for extraction of a distillate product sample from one or more locations along the processing line;
a cetane improver injection along the processing line; and
a plant distributed control system in electronic communication with at least the online DCN/ICN analyzer.

20. The distillate blending system of claim 19 wherein the one or more locations along the processing line for extraction of the distillate product sample includes a location upstream of the cetane improver injection and/or a location downstream of the cetane improver injection.
